(19) United States
(12) Patent Application Publication
(10) Pub. No.: US 2011/0306999 A1
(43) Pub. Date: Dec. 15, 2011

(54) STIMULATION OF SPECIFIC ACUPUNCTURE POINTS FOR VISION IMPROVEMENT

(75) Inventors: DENNIS Shun Chiu LAM, Kowloon (HK); Jianhao Zhao, Shantou City (CN); Lijia Chen, Jiangmen City (CN); Dorothy Shu Ping Fan, Hong Kong (HK); Johnson Yiu Nam Lau, Newport Beach, CA (US); Christopher Kai Shun Leung, Kowloon (HK); Mingzhi Zhang, Guangdong (CN); Chongren Zheng, Guangdong (CN); Andrew Chung Tin Fok, Kowloon (HK); Shirley Shu Liu, Dandong City (CN)

(73) Assignee: The Chinese University of Hong Kong, Sha Tin (CN)

(21) Appl. No.: 13/155,139

(22) Filed: Jun. 7, 2011

Related U.S. Application Data
(60) Provisional application No. 61/353,122, filed on Jun. 9, 2010.

(51) Int. Cl. A61H 99/00; A61B 17/34

(52) U.S. Cl. 606/189

(57) ABSTRACT

This invention relates to the stimulation of a specific combination of 5 acupuncture points including EX-HN5 (taiyang), BL59 (fuyang), BL2 (cuanzhu), GV20 (baihui), and LI4 (hegu) and its effectiveness in improving vision in the treatment of anisometropic amblyopia.

### RELATED APPLICATIONS

This application claims priority to U.S. Provisional Patent Application No. 61/353,122, filed Jun. 9, 2010, the contents of which are incorporated by reference in the entirety for all purposes.

### BACKGROUND OF THE INVENTION

This invention relates to the stimulation of a specific combination of various acupuncture points, including one that has never been used to treat eye diseases, to treat amblyopia (also known as lazy eye, a disorder of the visual system that is characterized by poor or lack of vision in an eye that is otherwise physically normal, or out of proportion to associated structural abnormalities). The fact that this unique combination of meridian points generating such a good clinical response was a surprise and has never been used or reported for the treatment of amblyopia.

Amblyopia is an important cause of visual impairment worldwide, with a prevalence of 0.3-5.0% (1-3). Over 50% of the patients with amblyopia are related to anisometropia (1, 2, 4). Treatment of anisometropic amblyopia is largely age-dependent. The international benchmark treatment of anisometropic amblyopia for children aged between 3 and 6 years comprises optical correction (e.g., wearing spectacles) alone. Approximately 77% of the treated patients showed good improvement after treatment (5). In older children aged between 7 and 12 years, however, only 30% responded to optical correction alone (6). Therefore, the international benchmark treatment for older children is a combination of optical correction and penalization of the better eye by occlusion or atropine treatment, which increased the response rate to 63% (6). However, reversed amblyopia (which refers to amblyopia that develops in the sound eye as a result of traditional amblyopia treatment, either patching [occlusion] or penalization [e.g., atropine] of the sound eye) is a potential complication of conventional patching. Moreover, poor compliance of patching will lead to a suboptimal treatment outcome while the use of atropine could also result in systemic toxicity and photophobia. Furthermore, it is not uncommon for patients to have a residual visual deficit. Alternative treatment for amblyopia is therefore warranted.

Acupuncture has been a recognized treatment over three thousand years. The exact mechanism of action has not been fully elucidated. It is believed that by targeting specific acupuncture points on the human body, therapeutic effects can be achieved through stimulation of the central and peripheral nervous system, and/or through other mechanisms. Nevertheless, the selection of acupuncture points varies from acupuncturist to acupuncturist.

Treating diseases using a combination of acupuncture points is an important concept in traditional Chinese medicine. More than 20 acupuncture points have been proposed to be useful to treat eye diseases and different combinations have been attempted (7-10). However, there is no strong evidence showing that a particular combination of acupuncture points is useful for the treatment of eye diseases.

Throughout their research, the present inventors identified, to their surprise, that by stimulating a combination of 5 specific acupuncture points in a pattern not previously recognized, acupuncture could greatly accelerate the treatment response and improve the overall treatment outcomes and shorten the total treatment duration in patients with amblyopia. The use of such a combination of acupuncture points has never been reported before. Preferably, the stimulation of these acupuncture points are simultaneous, typically starting at approximately the same time (i.e., the duration between the insertion of needles into the first and the last acupuncture points should be within 30-60 seconds) for approximately the same time duration (i.e., 15-20 minutes).

These acupuncture points are: EX-HN5 (taiyang), BL59 (fuyang), BL2 (cuanzhu), GV20 (baihui), and LI4 (hegu) (FIG. 1).

EX-HN5 is located at the temporal fossa and 1 cun behind the midpoint of a line from the lateral end of the eyebrow to the external canthus. BL59 is located at 3 cun above the site between the Achilles tendon and lateral malleolus. Stimulation at BL59 has only been reported in the treatment of acute lumbar sprain (11). Stimulation at BL59 has never been used to treat eye diseases or other human diseases other than acute lumber sprain. BL2 is located at the supraorbital notch and at the median end of the eyebrow. Stimulation at BL2 may increase blood flow velocity in the supratrochlear artery (12). GV20 resides at 7 cun above the occipital hairline and 5 cun behind the frontal hairline. Stimulation at BL2 and GV20 reduced P100 latencies in Visual Evoked Potential (VEP) tests (13). LI4 is located at the hand-dorsum and between the first and second metacarpals, midway down the second metacarpal. Stimulation at LI4 and GV20 may increase regional cerebral blood flow and glucose metabolism (14). None of the physiological changes reported by previous studies relating to these acupuncture points leads to the clinical implications relevant to this invention.

In a randomized controlled trial conducted by the inventors (15), the acupuncture points were stimulated in a group of 83 anisometropic amblyopic children aged between 3 and 6 years (FIG. 2). In the study, the participants were randomly divided into two groups in which they received different combination of treatment in the three different phases. In Phase I (Weeks 1-15), Group 1 (diamond line; n=42) received spectacle correction alone while Group 2 (square line; n=41) received spectacle correction and acupuncture. Treatment outcomes were measured at 5-week intervals in terms of improvement in best-corrected visual acuity (BCVA), which was expressed using logMAR scale, in the amblyopic eye. As the scale measures visual acuity loss, positive values indicate vision loss and 0.00 denotes normal vision. It should be noted that the lower the logMAR value, the better the visual acuity (FIGS. 2 and 3). It was found that Group 2 always demonstrated a statistically better improvement in BCVA than Group 1 at every interval in Phase I. Phase I of the study ended at week 15 as maximal improvement in visual acuity from spectacle correction was expected to be attained within the first 15 weeks of treatment (5, 16). Group 1 functioned as a control group that showed the treatment effect of spectacle correction. Additional improvement in BCVA demonstrated by Group 2 in Phase I should therefore be attributed to the use of acupuncture.

At week 16 when Phase II (Weeks 16-30) began, subjects in Group 1 were introduced to acupuncture in addition to spectacle correction, while subjects in Group 2 ceased to receive the acupuncture treatment and were treated with spectacle correction alone. It was expected that no or minimal additional improvement in visual acuity could be observed in subjects of Group 2 as they were not treated with acupuncture in Phase II and had already attained maximal treatment effect from spectacle correction in the first 15 weeks. The leveling-off of the curve, i.e. no more improvement in visual acuity, shown in the square line in Phase II had confirmed the expectation of the inventors. On the other hand, as subjects in Group 1 had already attained maximal improvement in visual acuity from spectacle correction in Phase I, any additional improvement obtained at the different intervals in Phase II could thus be attributable to the introduction of acupuncture. There were appreciable gains in mean BCVA in Group 1 at Weeks 20, 25 and 30 and such gains were statistically significant compared to the mean BCVA obtained at week 15 (P<0.0001), suggesting that acupuncture did have adjunctive effect to spectacle correction.

Both groups ceased to receive acupuncture in the last phase of the study (Phase III; weeks 31-60) in which they were treated with spectacle correction alone. It was found that the maximal treatment effect achieved earlier in Phase I and Phase II of the study stayed till the end of the study, indicating that there was no recurrence of amblyopia on the subjects. Therefore, the treatment effect of the combination of spectacle correction and acupuncture was believed to be sustainable.

The standard international treatment for anisometropic amblyopia in older children aged between 7 and 12 is a combination of spectacle correction and occlusion. In another randomized controlled trial conducted by the inventors (17), the effectiveness of treatment of anisometropic amblyopia with spectacle correction and acupuncture was compared with that of the conventional treatment which involves spectacle correction and 2-hour patching daily (FIG. 3). In the study, after having received spectacle correction for 16 weeks, 88 children aged between 7 and 12 years were randomly assigned to receive either two hours' of patching of the sound eye daily (diamond line) or five sessions of acupuncture weekly (circle line). Treatment outcomes were measured at 5-week intervals in terms of improvement in BCVA of the amblyopic-eye. BCVA was also expressed using logMAR scale and the treatment period lasted for 25 weeks. As the subjects had already received spectacle correction for 16 weeks, any treatment effect obtained during the study should primarily be attributable to acupuncture or patching. To the surprise of the inventors, subjects receiving acupuncture showed substantial improvement and their treatment outcomes were better than those receiving occlusion, as reflected by a comparatively steeper slope of some of the segments the circle line than those of the diamond line. Both the patching and the acupuncture groups demonstrated improvement in the BCVA at Weeks 5, 10 and 15, as reflected by the steady decline in the logMAR value. The curves of both the diamond and the circle lines leveled off between Weeks 15 and 25, due to the fact that maximal improvement in visual acuity was attained within the first 15 weeks of the study. The profile of the BCVA improvement obtained in the patching group of this study was similar to that documented in previous international studies (18, 19). Surprisingly, the 15-week mean BCVA of the acupuncture group (circle line) was 0.49 logMAR lines better than of the patching group (P=0.03). Therefore, the inventors believed that acupuncture was effective and had better results than conventional patching in treating amblyopia for children aged 7-12 and could thus be an alternative treatment to conventional patching for anisometropic amblyopia.

No side effects from acupuncture were encountered in both of the above trials.

### BRIEF SUMMARY OF THE INVENTION

According to the invention, acupuncture stimulation at a combination of certain specific acupuncture points, which includes the following 5 acupuncture points, namely GV20 (baihui), EX-HN5 (taiyang), BL2 (cuanzhu), LI4 (hegu) and BL59 (fuyang), is safe and effective in improving visual function in patients of amblyopia. The novelty of the present invention therefore resides in the recognition of using BL59 (an acupuncture point that has not been reported for treatment of eye diseases) and the specific combination with GV20 (baihui), EX-HN5 (taiyang), BL2 (cuanzhu), and LI4 (hegu) for the treatment of anisometropic amblyopia.

GV20 is located at 7 cun above the occipital hairline and 5 cun behind the frontal hairline. EX-NH5 is situated at the temporal fossa and 1 cun behind the midpoint of a line from the lateral end of the eyebrow to the external canthus. BL2 resides at the supraorbital notch and at the median end of the eyebrow. LI4 is located at the hand-dorsum and between the first and second metacarpals, midway down the second metacarpal. BL59 is found three cun above the site between the Achilles tendon and lateral malleolus.

The invention will be better understood by reference to the following detailed description in connection with the accompanying drawings.

### BRIEF DESCRIPTION OF THE DRAWINGS

FIG. 1 is a diagram showing the detailed locations of the 5 basic acupuncture points used in this invention.

FIG. 2 is a line graph illustrating the improvement in visual acuity after acupuncture (square line; Group 2) compared with that after spectacle correction alone (diamond line; Group 1) in children aged 3 to 6 years. The trials had 3 phases: 15 weeks for Phase I; another 15 weeks for Phase II; and 30 weeks for Phase III. After 15 weeks of treatment (Phase I), the best-corrected visual acuity (BCVA) had improved by a mean of 2.2 lines in Group 1 of which the subjects were treated with spectacle correction alone (diamond line) and 2.9 lines in Group 2 of which the subjects received acupuncture in addition to spectacle correction (square line). The visual acuity improvement in Group 2 was always better than that in Group 1 at all time points. After baseline adjustment, the mean difference in BCVA between the 2 groups was 0.70 (P=0.051), 0.81 (P=0.00019) and 0.77 lines (P=0.002) at Weeks 5, 10 and 15 respectively. After the regimens were crossed over in Phase II, i.e. subjects in Group 1 began to receive acupuncture in addition to spectacle correction while those in Group 2 ceased to receive acupuncture and were treated with spectacle correction alone, the 20-, 25- and 30-week mean BCVA in Group 1 had improved by 0.4 (P<0.0001), 0.7 (P<0.0001) and 1.2 (P=2×10⁻¹²) additional lines respectively, compared to the 15-week BCVA. The above additional improvement in BCVA should be attributed to the use of acupuncture as maximal improvement in visual acuity from spectacle correction was expected to be attained within the first 15 weeks of treatment. As subjects in Group 2 no longer received acupuncture in Phase II, no or minimal visual acuity improvement of 0 (P>0.5), 0 (P>0.1) and 0.4 (P=0.001) lines were obtained at the 20-, 25- and 30-week respectively. Such findings came as no surprise to the inventors as no acupuncture had been administered to the subjects in Group 2 in Phase II and maximal visual acuity improvement from spectacle correction was attained in Phase I. In Phase III, no acupuncture was administered to both groups. The leveling-off of the two lines in Phase III illustrated that there was no recurrence of amblyopia, which implied that the effects achieved by the combined treatment of acupuncture and spectacle correction received by both Groups 1 and 2 in Phase I and Phase II respectively were sustainable. Acupuncture was well tolerated with no major complications or side effects. Acupuncture is safe and well tolerated by children and provides good sustainable adjunctive effects on spectacle correction for both untreated and treated anisometropic amblyopia. Acupuncture accelerates the treatment response, improves the overall treatment results and shortens the total treatment duration.

FIG. 3 is a line graph illustrating the comparison between the improvement in visual acuity in children aged 7 to 12 years after the combined treatment of spectacle correction and acupuncture and that after conventional treatment that involved spectacle correction and patching. The use of spectacle correction along with patching or acupuncture was effective in treating anisometropic amblyopia in the first 15 weeks and both lines leveled off from week 16 to week 25. The mean BCVA of the amblyopic eye at week 15 improved from baseline by 1.83 and 2.27 lines in the patching and acupuncture groups respectively. After baseline adjustment, the mean BCVA obtained in the acupuncture group was better than that obtained in the patching group by an average of 0.34 (P=0.06), 0.31 (P=0.12) and 0.49 (P=0.03) logMAR lines at Weeks 5, 10 and 15 respectively. These results revealed that acupuncture produced better results than patching in the treatment of anisometropic amblyopia in children aged 7-12 years.

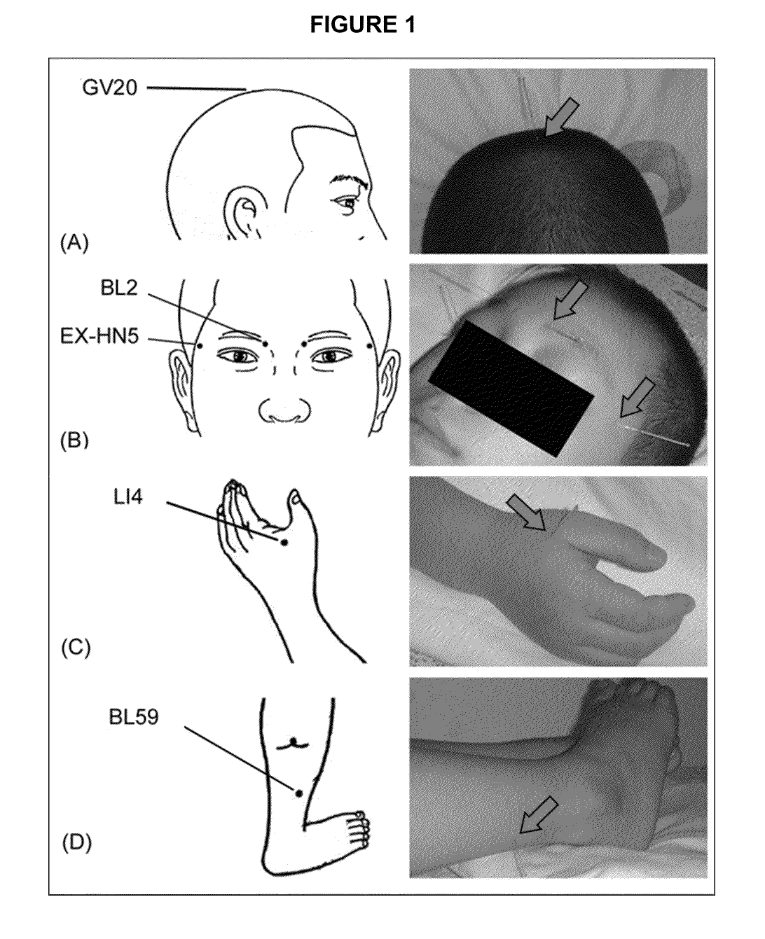

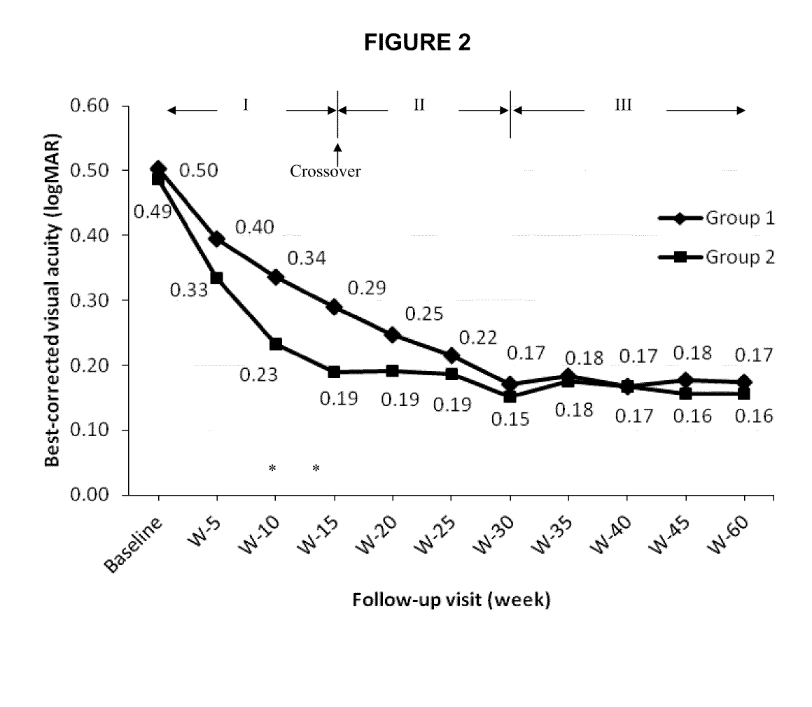

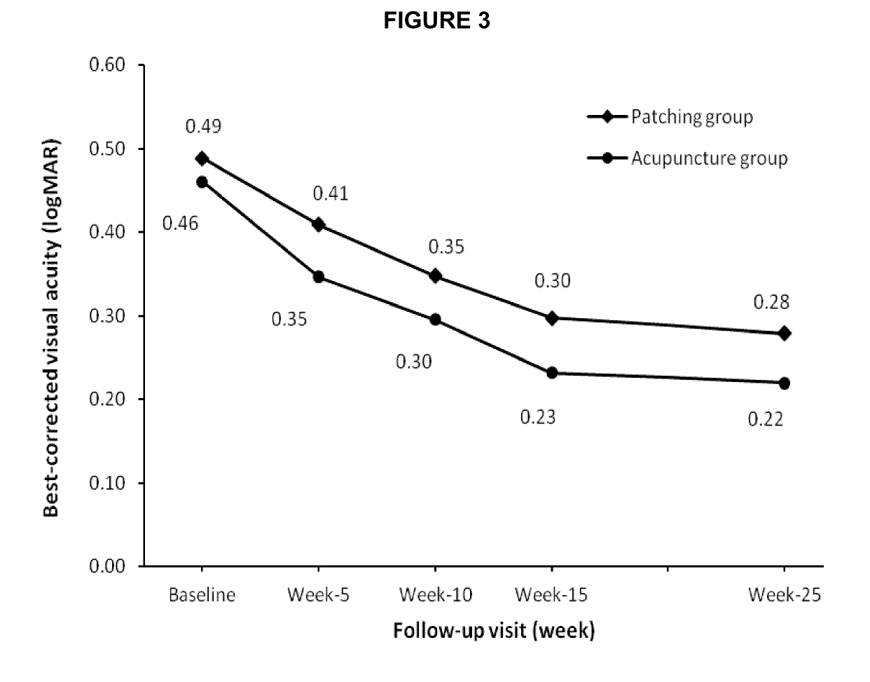

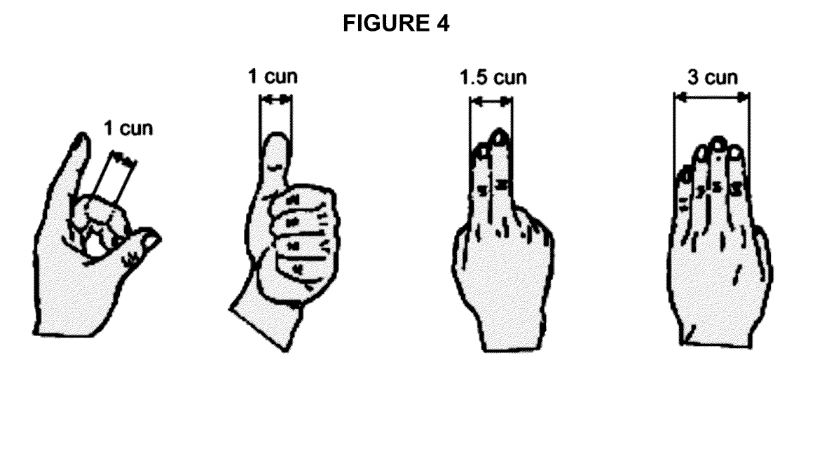

### DETAILED DESCRIPTION OF THE INVENTION

Acupuncture is administered 1-5 times per week for 3 months or more. Each treatment session lasts for at least 15 minutes. Five acupuncture points—GV20 (baihui), EX-HN5 (taiyang), BL2 (cuanzhu), LI4 (hegu) and BL59 (fuyang)—are stimulated. GV20 is located at 7 cun above the occipital hairline and 5 cun behind the frontal hairline. EX-NH5 is situated at the temporal fossa and 1 cun behind the midpoint of a line from the lateral end of the eyebrow to the external canthus. BL2 resides at the supraorbital notch and at the median end of the eyebrow. LI4 is located at the hand-dorsum and between the first and second metacarpals, midway down the second metacarpal. BL59 is found three cun above the site between the Achilles tendon and lateral malleolus.

Acupuncture points, or acupoints, in traditional Chinese medicine are locations on the body that are the focus of various treatment methods such as acupuncture, acupressure, sonopuncture, and laser acupuncture. There are several hundred acupoints that are distributed along meridians (which are connected points across the body that affect a specific organ or a body part), as well as numerous other “extra points” not associated with a particular meridian. The precise locations and nomenclature can be found in various publications including standard acupuncture nomenclature published by the World Health Organization (WHO) and the national standardized acupuncture point locations published by the government of the People's Republic of China (PRC). All acupuncture points used in practicing the present invention can be identified in accordance with the standardized nomenclature published by the WHO or the PRC government.

Cun Measurements of the Body: The “cun” is a unit of length widely used in acupuncture and traditional Chinese medicine. Taking individual differences into account, the “cun” can measure and locate accurately acupoints on any individual's body with the help of a person's inborn measurement system, which facilitates the finding of the points. The process starts with the measurement of one cun. This can be done in two ways: (a) using the width of the distal inter-phalangeal joint of the thumb (first finger) or (b) using the distance between the distal and proximal inter-phalangeal joints of the 3^{rd} (middle) finger. All other specific measurements are outlined in the diagrams below. When in doubt, the thumb (1 cun) or the four finger method (3 cun) can always be referred to. FIG. 4 is an illustration taken from the “tcmstudent” website.

Stimulation at the acupuncture points can be achieved via inserting sterilized disposable steel needles, e.g. a 32-gauge stainless steel, into the skin. The depths of needle insertion for GV20, EX-HN5, BL2, LI4 and BL59 are 1-2 mm, 6-10 mm, 2-6 mm, 10-15 mm, and 10-20 mm, respectively.

The needles were manually manipulated by rotation to achieve “de qi,” which is a unique sensation that indicates the efficacy of acupuncture treatment. It is contemplated that this may also be accomplished by electric impulse stimulation and/or other various forms of stimulation including electromagnetic manipulations, laser, and/or acupressure techniques known to those of skill in the art.

The needles are then left in place after insertion for a sufficient amount of time such that the stimulation will be effective for the intended therapeutic purpose. Typically, the needles are left in place for a duration of at least 15 or 20 minutes after insertion of the last needle. The acupuncturist revisits and manipulates the needles where necessary.

Alternatively, the stimulation can be accomplished by electric impulse stimulation and/or other various forms of stimulation including electromagnetic manipulations, laser, and/or acupressure techniques. Manual manipulation by human hands applying effective amount of pressure at these points are also useful stimulation for the intended therapeutic purpose, and the manipulation may include primarily downward pressure application and optionally in combination with horizontally directional motions, such as a rotational motion. Typically, the time duration for these alternative methods of stimulation is also at least 15 or 20 minutes.

Regardless of the mode of stimulation, treatment time and/or intensity should be adequate for the particular mode chosen, such that an effective amount of stimulation is administered to the patient being treated. As used in this disclosure, the term “effective amount” refers to an amount of treatment, which may be manifested in the time duration of the treatment (e.g., leaving needles in place for at least 15-20 minutes) and/or intensity of the treatment (e.g., the amount of pressure or electrical current applied to the acupuncture points), that is adequate for the intended therapeutic effects to be achieved (e.g., the patient's vision is improved, or the symptoms of anisometropic amblyopia are ameliorated).

At GV20, EX-HN5 and BL2, the directions of needle insertion can be perpendicular or oblique. At LI4 and BL59, the directions of needle insertion are perpendicular and oblique respectively.

All patents, patent applications, and other publications cited in this application are incorporated by reference in their entirety for all purposes.

### LIST OF REFERENCES

1. Attebo K, Mitchell P, Cumming R, Smith W, Jolly N, Sparkes R. Prevalence and causes of amblyopia in an adult population. Ophthalmology 1998; 105(1):154-9.
2. Rosman M, Wong T Y, Koh C L, Tan D T. Prevalence and causes of amblyopia in a population-based study of young adult men in Singapore. Am J Ophthalmol 2005; 140(3):551-2.
3. Holmes J M, Clarke M P. Amblyopia. Lancet 2006; 367(9519):1343-51.
4. Pediatric Eye Disease Investigator Group. The clinical profile of moderate amblyopia in children younger than 7 years. Arch Ophthalmol 2002; 120(3):281-7.
5. Cotter S A, Edwards A R, Wallace D K, Beck R W, Arnold R W, Astle W F, Barnhardt C N, Birch E E, Donahue S P, Everett D F, Felius J, Holmes J M, Kraker R T, Melia M, Repka M X, Sala N A, Silbert D I, Weise K K. Treatment of anisometropic amblyopia in children with refractive correction. Ophthalmology 2006; 113(6):895-903.
6. Scheiman M M, Hertle R W, Beck R W, Edwards A R, Birch E, Cotter S A, Crouch E R, Jr., Cruz O A, Davitt B V, Donahue S, Holmes J M, Lyon D W, Repka M X, Sala N A, Silbert D I, Suh D W, Tamkins S M. Randomized trial of treatment of amblyopia in children aged 7 to 17 years. Arch Ophthalmol 2005; 123(4):437-47.
7. Shin M S, Kim J I, Lee M S, Kim K H, Choi J Y, Kang K W, Jung S Y, Kim A R, Kim T H. Acupuncture for treating dry eye: a randomized placebo-controlled trial. Acta Ophthalmol 2010; 88(8):e328-33.
8. Kurusu M, Watanabe K, Nakazawa T, Seki T, Arai H, Sasaki H, Fuse N, Tamai M. Acupuncture for patients with glaucoma. Explore (NY) 2005; 1(5):372-6.
9. Tseng K L, Liu H J, Tso K Y, Woung L C, Su Y C, Lin J G. A clinical study of acupuncture and SSP (silver spike point) electro-therapy for dry eye syndrome. Am J Chin Med 2006; 34(2):197-206.
10. Nepp J, Jandrasits K, Schauersberger J, Schild G, Wedrich A, Sabine G L, Spacek A. Is acupuncture an useful tool for pain-treatment in ophthalmology? Acupunct Electrother Res 2002; 27(3-4):171-82.
11. Hu R. Treatment of acute lumbar sprain with acupuncture at Fuyang (UB 59). J Tradit Chin Med 1993; 13(4):264-5.
12. Litscher G, Wang L, Yang N H, Schwarz G. Ultrasound-monitored effects of acupuncture on brain and eye. Neurol Res 1999; 21(4):373-7.
13. Sagara Y, Fuse N, Seimiya M, Yokokura S, Watanabe K, Nakazawa T, Kurusu M, Seki T, Tamai M. Visual function with acupuncture tested by visual evoked potential. Tohoku J Exp Med 2006; 209(3):235-41.
14. An Y S, Moon S K, Min I K, Kim D Y. Changes in regional cerebral blood flow and glucose metabolism following electroacupuncture at LI 4 and LI 11 in normal volunteers. J Altern Complement Med 2009; 15(10):1075-81.
15. Lam D S, Zhao J, Chen L J, Wang Y, Zheng C, Lin Q, Rao S K, Fan D S, Zhang M, Leung P C, Ritch R. Adjunctive Effect of Acupuncture to Refractive Correction on Anisometropic Amblyopia One-Year Results of a Randomized Crossover Trial. Ophthalmology 2011.
16. Stewart C E, Moseley M J, Fielder A R, Stephens D A. Refractive adaptation in amblyopia: quantification of effect and implications for practice. Br J Ophthalmol 2004; 88:1552-6.
17. Zhao J, Lam D S, Chen L J, Wang Y, Zheng C, Lin Q, Rao S K, Fan D S, Zhang M, Leung P C, Ritch R. Randomized controlled trial of patching vs acupuncture for anisometropic amblyopia in children aged 7 to 12 years. Arch Ophthalmol 2010; 128(12):1510-7.
18. Wallace D K, Edwards A R, Cotter S A, et al. A randomized trial to evaluate 2 hours of daily patching for strabismic and anisometropic amblyopia in children. Ophthalmology. 2006; 113(6):904-912.
19. Scheiman M M, Hertle R W, Kraker R T, et al. Patching vs atropine to treat amblyopia in children aged 7 to 12 years: a randomized trial. Arch Ophthalmol. 2008; 126(12):1634-1642.

What is claimed is:

1. A method for improving a patient's vision comprising the step of simultaneously stimulating multiple acupuncture points comprising GV20 (baihui), EX-HN5 (taiyang), BL2 (cuanzhu), LI4 (hegu), and BL59 (fuyang) on the patient.

2. The method of claim 1, wherein said stimulating is achieved by inserting acupuncture needles at the acupuncture points to a depth of 1- to 20 mm.

3. The method of claim 1, wherein said stimulating is achieved by manual manipulation, electric impulse stimulation, electromagnetic manipulations, laser, or acupressure techniques.

4. The method of claim 3, wherein said manual manipulation comprises rotational motion.

5. The method of claim 2, wherein said needles are left at the acupuncture points for at least 15 minutes.

6. The method of claim 1, wherein the patient suffers from anisometropic amblyopia prior to the treatment.
